(19) United States
(12) Patent Application Publication
(10) Pub. No.: US 2008/0227701 A1
(43) Pub. Date: Sep. 18, 2008

(54) ANTIANGIOGENIC POLYPEPTIDES AND METHODS FOR INHIBITING ANGIOGENESIS

(76) Inventors: Jack HENKIN, Highland Park, IL (US); Donald J. DAVIDSON, Gurnee, IL (US)

Correspondence Address: DYKEMA GOSSETT PLLC, 10 S. WACKER DR., STE. 2300, CHICAGO, IL, 60606, US

(21) Appl. No.: 11/829,778

(22) Filed: Jul. 27, 2007

Related U.S. Application Data
(63) Continuation of application No. 10/778,423, filed on Feb. 13, 2004, now abandoned.
Application No. 10/778,423 is a continuation of application No. 09/967,386, filed on Sep. 28, 2001, now abandoned.
(60) Provisional application No. 60/236,550, filed on Sep. 29, 2000.

(51) Int. Cl. A61K 38/16; C07K 14/00; A61P 35/00; C07K 7/06; C07K 7/08; A61K 38/10; A61K 38/08; C08G 65/34

(52) U.S. Cl. 514/12; 530/350; 530/330; 530/324; 530/329; 514/18; 514/17; 514/16; 528/425

(57) ABSTRACT

Conjugated kringle protein fragments are disclosed as compounds for treating angiogenic diseases. Methods and compositions for inhibiting angiogenic diseases are also disclosed.

### CROSS-REFERENCE TO RELATED APPLICATIONS

This application claims priority to U.S. Provisional Patent Application Ser. No. 60/236,550, filed Sep. 29, 2000, which is hereby incorporated by reference.

### TECHNICAL FIELD

The present invention relates to the field of peptide chemistry. More particularly, the invention relates to the preparation and use of conjugated peptides containing amino acid sequences substantially similar to the corresponding sequences of mammalian plasminogen, pharmaceutical compositions containing the peptides, and treatment of diseases which arise from or are exacerbated by angiogenesis.

### BACKGROUND OF THE INVENTION

Angiogenesis, the process by which new blood vessels are formed, is essential for normal body activities including reproduction, development and wound repair. Although the process is not completely understood, it is believed to involve a complex interplay of molecules which regulate the growth of endothelial cells (the primary cells of capillary blood vessels). Under normal conditions, these molecules appear to maintain the microvasculature in a quiescent state (i.e. one of no capillary growth) for prolonged periods which may last for as long as weeks or, in some cases, decades. When necessary (such as during wound repair), these same cells can undergo rapid proliferation and turnover within a 5 day period Folkman, J. and Shing, Y., The Journal of Biological Chemistry, 267(16), 10931-10934, and Folkman, J. and Klagsbrun, M., Science, 235, 442-447 (1987)).

Although angiogenesis is a highly regulated process under normal conditions, many diseases (characterized as angiogenic diseases) are driven by persistent unregulated angiogenesis. Otherwise stated, unregulated angiogenesis may either cause a particular disease directly or exacerbate an existing pathological condition. For example, ocular neovascularization has been implicated as the most common cause of blindness and dominates approximately 20 eye diseases. In certain existing conditions, such as arthritis, newly formed capillary blood vessels invade the joints and destroy cartilage. In diabetes, new capillaries formed in the retina invade the vitreous, bleed, and cause blindness. Growth and metastasis of solid tumors are also dependent on angiogenesis Folkman, J., Cancer Research, 46, 467-473 (1986), Folkman, J., Journal of the National Cancer Institute, 82, 4-6 (1989)). It has been shown, for example, that tumors which enlarge to greater than 2 mm must obtain their own blood supply and do so by inducing the growth of new capillary blood vessels. Once these new blood vessels become embedded in the tumor, they provide a means for tumor cells to enter the circulation and metastasize to distant sites such as liver, lung or bone (Weidner, N., et al, The New England Journal of Medicine, 324(1): 1-8 (1991)).

Several angiogenesis inhibitors are currently under development for use in treating angiogenic diseases, but there are disadvantages associated with these compounds. Fumagillin, a compound secreted by the fungus Aspergillus fumigatis fresenius, has demonstrated angioinhibitory effects, but has not been developed clinically due to the dramatic weight loss suffered by laboratory animals after prolonged exposure. TNP-470, a synthetic analog of fumagillin, also inhibits endothelial growth, but has been shown to induce asthenial and neurocortical toxicity in humans, limiting allowable dosages (J. Clin. Oncology 17, 2541 (1989)).

To date, several angiogenic factors, specifically kringle peptide fragments of plasminogen have been described (see, for example, U.S. Pat. No. 5,639,725; U.S. Pat. No. 5,733,876; U.S. Pat. No. 5,792,845; U.S. Pat. No. 5,837,682; U.S. Pat. No. 5,861,372; U.S. Pat. No. 5,885,795; U.S. Pat. No. 6,024,688; U.S. Pat. No. 5,854,221, U.S. Pat. No. 5,801,146; U.S. Pat. No. 5,981,484; U.S. Pat. No. 6,057,122; and U.S. Pat. No. 5,972,896, which are incorporated herein by reference in their entirety). Despite the promising anti-angiogenic activity of these fragments, their circulation half-lives are short. Thus, there is still a need for anti-angiogenic compounds, such as kringle peptide fragments, which have improved pharmacokinetic activity. Such compounds should also be easily and cost-effectively made.

### SUMMARY OF THE INVENTION

In its principle embodiment, the present invention discloses a conjugated kringle peptide fragment consisting of a functionalized kringle peptide fragment chemically coupled to a functionalized polymer. Preferably, the N-terminus of the functionalized kringle peptide fragment is conjugated to the functionalized polymer through either an oxime bond or through a carbon-nitrogen single bond.

In a preferred embodiment, the functionalized kringle peptide fragment consists essentially of a kringle peptide fragment selected from the group consisting of kringle 1 of plasminogen, kringle 5 of plasminogen, kringles 4-5 of plasminogen, and kringle 2 of prothrombin. Preferably, the kringle peptide fragment is kringle 5 of plasminogen or kringles 4-5 of plasminogen. Most preferably, the kringle peptide fragment is a kringle 5 peptide fragment that has substantial sequence homology to a plasminogen fragment selected from the group consisting of human, murine, bovine, canine, feline, Rhesus monkey, and porcine plasminogen. In another preferred embodiment, the functionalized polymer consists essentially of a polymer which is a polyalkylene glycol. Preferably the polyalkylene glycol is selected from the group consisting of straight, branched, disubstituted, or unsubstituted polyalkylene glycol, polyethylene glycol homopolymers, polypropylene glycol homopolymers, and copolymers of ethylene glycol with propylene glycol, wherein said homopolymers and copolymers are unsubstituted or substituted at one end with an alkyl group. Particularly preferred polyalkylene glycols are polyethylene glycol (PEG) and methoxypolyethylene glycol (mPEG) having molecular weights of about 5,000 to about 40,000. Most preferably, the polyalkylene glycol is methoxypolyethylene glycol (mPEG) having a molecular weight of about 10,000 to about 20,000.

In another embodiment, the present invention discloses a pharmaceutical composition comprising a conjugated kringle peptide fragment in combination with a therapeutically acceptable carrier.

In another embodiment, the present invention discloses a method of treating a disease in a patient in need of anti-angiogenic therapy comprising administering to a human or animal a therapeutically effective amount of a conjugated kringle peptide fragment. Preferably, the disease is selected from the group consisting of cancer, arthritis, macular degeneration, and diabetic retinopathy. More preferably, the disease is cancer, and most preferably, the disease is selected from the group consisting of primary and metastatic solid tumors, carcinomas, sarcomas, lymphomas, psoriasis, and hemagiomas.

In another embodiment, the present invention discloses a method of inhibiting endothelial cell proliferation in an individual comprising administering to said individual an effective amount of a conjugated kringle peptide fragment.

In another embodiment, the present invention discloses a method of inhibiting endothelial cell proliferation in vitro comprising administering to an endothelial cell an effective amount of a conjugated kringle peptide fragment.

### DETAILED DESCRIPTION OF THE INVENTION

As used herein, the singular forms “a”, “an”, and “the” include plural reference unless the context clearly dictates otherwise. In addition, all published scientific and patent literature (including patent applications) referred to herein are hereby incorporated by reference in their entirety.

As used herein, the terms “K5”, “Kringle 5”, “K1”, and “Kringle 1” refer to kringle regions of plasminogen.

As used herein, the term “K2” refers to a kringle region of prothrombin.

Unless otherwise stated, the following terms shall have the following meanings:

The term “acyl group” means a C₁-C₁₀ alkyl group attached to the parent molecular moiety through a carbonyl group.

The term “acylating group” means a reagent capable of donating an acyl group to the nitrogen atom of a molecule during the course of a reaction. Examples of acylating groups include acetic anhydride, acetyl chloride, and the like.

The term “C₁-C₁₀ alkyl” means a monovalent group of one to ten carbon atoms derived from a straight or branched chain saturated hydrocarbon by the removal of a single hydrogen atom.

The term “C₁-C₁₀ alkylene” means a divalent group of one to ten carbon atoms derived from a straight or branched chain saturated hydrocarbon. The term “carbonyl group” means —C(O)—.

The term “conjugate of a kringle peptide fragment” or “conjugated kringle peptide fragment” means a functionalized kringle peptide fragment chemically coupled to a functionalized polymer. Methods of making such conjugates have been described (see, for example, WO96/41813 and J. Pharmaceut. Sci. 87, 1446-1449 (1998)). Non-limiting examples of conjugates of kringle peptide fragments include a kringle 5 peptide fragment coupled to polyethylene glycol, a kringle 5 peptide fragment coupled to methoxypolyethylene glycol, a kringle 4-5 peptide coupled to methoxypolyethylene glycol, and the like.

The term “functionalized kringle peptide fragment” refers to a kringle peptide fragment, as defined herein, which has been modified site-specifically to contain a reactive group. Functionalized kringle peptide fragments also are intended to include kringle peptide fragments which have been modified by the addition of one or more non-native amino acids at the N-terminus. The additional amino acid may serve as the reactive group or, alternatively, may itself be further modified to form a reactive group. For example, a serine (Ser) may be added immediately upstream (i.e., before) the leucine (Leu) residue at position 450 of a kringle 5 peptide fragment having the sequence from amino acid position 450 to amino acid position 543. The N-terminal Ser residue may then be specifically modified to form a reactive group such as an aldehyde. Kringle peptide fragments having one or more non-native amino acids at their N-terminus can be made by any means well known in the art, such as, for example, by recombinant DNA methodologies or by synthetic peptide chemistry. Means for functionalizing polypeptides are known to those of ordinary skill in the art (see, for example, WO 96/41813 and J. Pharmaceut. Sci. 87, 1446-1449 (1998)).

The term “functional or functionalized polymer” means the formation of either an amino-oxy group or an aldehyde on a polymer, as defined herein, so that the polymer can be site-specifically conjugated to a complementary functionalized target, such as a functionalized kringle peptide fragment. By way of example, means to functionalize polymers also are provided in WO96/41813 and J. Pharmaceut. Sci. 87, 1446-1449 (1998)).

The term “kringle fusion protein” refers to a polypeptide comprising an amino acid sequence drawn from two or more individual polypeptides, one of which is a kringle peptide fragment. A kringle fusion protein may be formed by the expression of a polynucleotide in which the coding sequence for a kringle peptide fragment has been joined with the coding sequence of at least one other polypeptide such that the two (or more) reading frames are in frame. Non-limiting examples of kringle fusion proteins include fusions of two or more individual kringle peptide fragments such as kringles 4-5 (K4-5). Other examples of kringle fusion proteins of the present invention include a kringle peptide fragment alone or fused with another polypeptide further joined to a biological tag. Such kringle fusion proteins may or may not be capable of being cleaved into the separate proteins from which they are derived.

The term “kringle peptide fragment” refers to a kringle region of plasminogen or prothrombin having anti-angiogenic activity and includes a sequence of: (a) an individual kringle peptide such as kringle 1 (K1) and kringle 5 (K5) of plasminogen; (b) a kringle peptide such as kringle 2 (K2) of prothrombin; (c) a kringle fusion protein as defined herein; (d) a kringle peptide having substantial sequence homology to the sequences of (a), (b), or (c); and (e) a peptide segment of (a), (b), or (c) which has substantial sequence homology to the sequences described in (a), (b), and (c). The aforementioned kringle peptide fragments and methods for making and using said fragments have been extensively described in the patent literature (see U.S. Pat. No. 5,639,725; U.S. Pat. No. 5,733,876; U.S. Pat. No. 5,792,845; U.S. Pat. No. 5,837,682; U.S. Pat. No. 5,861,372; U.S. Pat. No. 5,885,795; U.S. Pat. No. 6,024,688; U.S. Pat. No. 5,854,221, U.S. Pat. No. 5,801,146; U.S. Pat. No. 5,981,484; U.S. Pat. No. 6,057,122; and U.S. Pat. No. 5,972,896) as well as in the scientific literature. A preferred kringle peptide fragment is a K5 peptide fragment, either alone, fused with one or more kringle peptide fragments or used in combination with other kringles regions of plasminogen (e.g., angiostatin) and/or anti-angiogenic agents, such as endostatin. Yet another preferred kringle peptide fragment is one having substantial sequence homology to a human K5 peptide fragment, as described herein. Particularly preferred K5 peptide fragments have the sequences from amino acid residue 450 to 543 of SEQ ID NO: 1 and from amino acid residue 458-543 of SEQ ID NO: 1.

The term “polymer” means a chemical compound consisting of repeating non-peptide structural units. Preferred polymers of the present invention are water-soluble. Most preferred water-soluble polymers are polyethylene glycol (PEG) and methoxypolyethylene glycol (mPEG).

The terms “polypeptide”, “peptide”, and “protein” each refer to a molecular chain of amino acids and does not refer to a specific chain length. This term is also intended to refer to post-expression modifications of the polypeptides, peptides, and proteins, for example, glycosylations, acetylations, phosphorylations and the like.

The term “purified polypeptide” means a polypeptide of interest or fragment thereof which is essentially free, that is, contains less than about 50%, preferably less than about 70%, and more preferably, less than about 90% of cellular components with which the polypeptide of interest is naturally associated. Methods for purifying polypeptides are well known in the art.

The term “substantial sequence homology” means approximately 60% amino acid identity, desirably at least approximately 70% amino acid identity, more desirably approximately 80% amino acid identity, even more desirably approximately 90% identity and most desirably approximately 95% amino acid identity of the corresponding peptide sequence of human, murine, bovine, canine, feline, Rhesus monkey, or monkey plasminogen. Sequences having substantial sequence homology to human plasminogen are referred to as “homologues”. In addition to having substantial sequence homology, homologues of the present invention demonstrate like biological activity (i.e., anti-angiogenic activity) as kringle peptide fragments described herein. Because the amino acid sequence or the number of amino acids in a kringle peptide fragment may vary from species to species or from the method of production, the total number of amino acids in a kringle peptide fragment cannot, in some instances, be defined exactly. Given that these sequences are identical in at least 73% of their amino acids, it is to be understood that the amino acid sequence of a kringle peptide fragment is substantially similar among species and that methods of production of kringle peptide fragments provide kringle peptide fragments with substantial sequence homology to the corresponding amino acid sequences of human plasminogen.

All peptide sequences are written according to the generally accepted convention whereby the a-N-terminal amino acid residue is on the left and the a-C-terminal is on the right. As used herein, the term “a-N-terminal” refers to the free alpha-amino group of an amino acid in a peptide, and the term “a-C-terminal” refers to the free alpha-carboxylic acid terminus of an amino acid in a peptide.

The present invention provides compounds having anti-angiogenic activity comprising a functionalized kringle peptide fragment conjugated to a functionalized polymer. The formation of such conjugates renders them more suitable as therapeutic agents. For example, desirable properties of conjugated kringle peptide fragments include increased solubility in aqueous solutions, increased stability during storage, reduced immunogenicity, increased resistance to enzymatic degradation, compatibility with a wider variety of drug administration systems, and increased in vivo half-lives.

In one embodiment, functionalized polymers of the invention are represented by the following formula:

P-X—O—NH₂

wherein P represents a polymer, preferably water soluble, X represents a spacer group which is optionally present, and —O—NH₂ represents amino-oxy. As the polymer P comprises multiple repeating units of varying amounts, it will be appreciated that the molecular weight of P may vary considerably. When P is said to have a given molecular weight, that molecular weight may be only an approximation, reflecting the average molecular weight of a population of P molecules which differ from one another in the number of subunits present in the molecule. Generally, P will have a molecular weight of about 5,000 to about 40,000 and preferably from about 10,000 to about 20,000. It is to be understood, however, that the molecular weights which are suitable for P will vary depending upon the kringle peptide fragment to be modified.

The spacer group X may be absent or present and if present, functions to connect an amino-oxy group to the polymer of interest. The spacer group X represents a non-reacting group comprising substituted or unsubstituted, branched or liner, aliphatic or aromatic groups such as phenyl or C₁-C₁₀ alkylene moieties, C₁-C₁₀ alkyl groups or a combination thereof, or an amino acid chain (such as a flexible hinge or loop sequence (see, for example, J. Mol. Biol., 211, 943-958 (1990)), a nucleotide chain, a sugar chain, a lipid chain, or a combination thereof and may contain heteroatoms. In the preferred embodiments, X comprises —CH₂— or —CHOH— or —COCH₂— or —NH— CO—CH₂—. When an amino-oxy group is on the functionalized polymer, groups present in the additional connecting structure (i.e., spacer groups) adjacent to the amino-oxy function are not critical; however, a requirement of any spacer group is that it not interfere with the formation of the oxime linkage between the amino-oxy group and its complementary aldehyde. Furthermore, spacer groups should not react in preference to the amino-oxy group with the aldehyde, nor provide steric hindrance to the reaction, nor deactivate the reactive groups.

In another embodiment, bi- and multi-polymer-containing functionalized polymers are provided. Such polymers have the general formula:

(P)ₘ-L-X—O—NH₂

wherein P, X, and O—NH₂ are as defined herein, m is an integer from 2 to 10, more preferably 2 to 5, and L is a multivalent linking group to which each P (m in number) is separately and covalently linked, and wherein the valency of L is at least m+1. In the case of bi- and multi-polymer functionalized polymers of the invention, conjugation of the functionalized polymer site-specifically to a target macromolecule results in attachment of two or more polymers site-specifically through a single oxime linkage via the —O—NH₂ group attached to the multivalent L structure.

Accordingly, the bi- or multi-polymer-containing functionalized polymer enables two or more polymers, the same or different, preferably the same, to be attached to a single, pre-chosen site on the target macromolecule. Where L is a trivalent group, m is 2. Preferably, the valence of L is m+1, wherein one valency of L is occupied by the oxime-forming group optionally through X, and the remaining valencies of L are occupied by one or more (i.e., m) polymers. The structure of L is not critical nor are the linkages connecting L to the polymers so long as L provides no steric hindrance to the subsequent oxime reaction, nor deactivate the reactive groups. L does not react with other functions present. Each arm or valency of the linking group L in the functionalized polymer preferably comprises a non-reacting group comprising substituted or unsubstituted aliphatic or aromatic groups such as phenyl or C₁-C₁₀ alkylene moieties, C₁-C₁₀ alkyl groups, or a combination thereof, or an amino acid chain such as a flexible hinge or loop sequence (such as a flexible hinge or loop sequence (see, for example, J. Mol. Biol., 211, 943-958 (1990)), a nucleotide chain, a sugar chain, a lipid chain, or a combination thereof and may contain heteroatoms. Prior to conjugation with a polymer, preferably all but one arm or valency of L contains a functional group that can react specifically with a group on the polymer, which preferably is located at a polymer terminus. The remaining valency is protected for later reaction with, or otherwise occupied with, a compound providing the oxime-forming function (said function is in a deprotecable state if desired). Where the polymer conjugate is to be used for antigenic or immunogenic purposes, it is apparent to one skilled in the art that linker groups are chosen that are not themselves strongly immunogenic. Where the polymer conjugate is to be used for binding purposes, the preferred linker group enhances or at least does not interfere with properties such as binding, avidity, product stability, or solubility. Linking structures can themselves contain valencies occupied with oxime-forming groups such that parallel assembly via oxime formation with a complementary functionalized polymer of the invention is employed to assemble the (P)ₘL-structure. Accordingly, baseplate structures described in U.S. Ser. Nos. 08/057,594, 08/114,877, and 08/057,594, and International application PCT/IB94/00093 are suitable for use as L structures. The oxime-forming groups of the baseplates can be replaced with other complementary reactive groups; however, most preferably oxime formation is used for assembly. A preferred L structure is derived from a tri-amine compound wherein any two amino groups are each available for coupling to a polymer and the remaining amino group is available for introduction of an oxime-forming group. A preferred tri-amine is a compound of the formula N(R⁵—NH₂)₃, wherein any two amino groups (—NH₂) are available for coupling to the polymer and the remaining amino group is available for introduction of an oxime-forming group, and R⁵ is a non-reacting group comprising substituted or unsubstituted aliphatic or aromatic groups such as phenyl or C₁-C₁₀ alkylene moieties, C₁-C₁₀ alkyl groups, or a combination thereof, or an amino acid chain (such as a flexible hinge or loop sequence (see, for example, J. Mol. Biol., 211, 943-958 (1990)), a nucleotide chain, a sugar chain, a lipid chain, or a combination thereof and may contain heteroatoms. R⁵ is preferably —CH₂—CH₂—. The three primary amino groups are preferably distal to the nitrogen. Most preferably the tri-amine compound is tris-(2-aminoethyl)amine.

In another embodiment, bi- or multi-polymer functionalized polymers are synthesized by first obtaining an L structure of desired multi-valency, usually having one valency protected, and then reacting the protected L-structure with an appropriately activated polymer intermediate typically using linking chemistries known in the art or with a functionalized polymer of the invention via oxime chemistry. After isolation of the bi- or multi-polymer product, the product is functionalized according to the invention by deprotection of the protected remaining valency of L followed by subsequent reaction (e.g., acylation in the case of an amino group) with a suitably protected amino-oxy containing acylating group. After deprotection of the oxime-forming functional group, the final product, the bi-or multi-polymer functionalized polymer is obtained.

Alternatively, L is first derivatized with a suitably protected-amino-oxy containing group, the mono-substituted L derivative is then reacted at each remaining valency with a polymer intermediate (such as one having a COOH when L contains NH₂ or an NH₂ group when L contains COOH) or with a functionalized polymer of the invention (when oxime chemistry is used to assemble P to L). For example, mPEG-COOH intermediate polymers can be coupled to free amino groups on an L-structure in the presence of HOBt (1-hydroxybenzotriazole hydrate) and DCC (1,3-dicyclohexylcarbodiimide), or without these reagents if the succinimidyl derivative of mPEG-COOH is previously prepared. After deprotection of the oxime-forming functional group, the final product, the bi- or multi-polymer functionalized polymer is obtained.

When the L group is formed from amino acids, the peptide sequence of an L structure can be synthesized by routine solid phase peptide synthesis (“SPPS”), and while the peptide is still attached to the solid phase PEG-COOH in an activated form, such as the N-hydroxysuccinimide ester, can be added to the nascent peptide chain. For example, the L structure can consist of a peptide having six reactive groups such as five lysine residues and an N-terminal amino group. PEG-COOSu hydroxysuccinimide ester can react with each of the s-amino groups of the lysine residues (while the N-terminus α-amino group is left protected). The N-terminal amine group of the fully acylated peptide is then unprotected and the polymer-containing structure is reacted with Boc-AoA-containing active ester to introduce the AoA group, which after Boc removal and mild cleavage from the resin, yields a penta-polymer-containing functionalized polymer of the invention. It is noteworthy that this method finds particular use with synthetic structures (and perhaps certain recombinant products) since these can be designed to exclude additional residues, that would require protection during the process and deprotection afterwards. Alternatively, Boc-Ser(benzyl)-OH or Boc-Ser(t-butyl)-OH in an activated form, such as the N-hydroxysuccinimide ester, can be attached to the ε-amino groups of the lysine residues. The N-terminus α-amino is then deprotected to introduce an amino-oxy group (e.g., AoA) so that after Boc removal a precursor L structure containing ε-Ser-pentalysine is obtained. Treatment of the precursor L structure once conjugated to the protein with a mild oxidizing agent, such as periodate at pH 7, will convert ε-Ser-pentalysine to ε-GXL-pentalysine, thus producing a penta-GXL L structure that can then be reacted with an amino-oxy functionalized polymer of the invention. The oxidation reaction can be terminated using any 1,2-diol or l-amino-2-ol or l-ol-2-amino compound having relatively free rotation about the 1,2 bond, such as ethylene glycol. Alternatively, the oxidation reaction can be terminated by rapid removal of the periodate, for example by reverse phase high performance liquid chromatography (RP-HPLC). Since the oxidation reaction only occurs with serine residues containing a primary amino group, only the e-serine residues are converted to the glyoxyl. One skilled in the art knows of methods for chemically protecting an N-terminal serine from oxidation, when protection is desired. The N-terminal amine group can then be unprotected and the polymer-containing structure is reacted with a Boc-AoA-containing active ester to introduce the AoA group, which after Boc removal, yields a penta-polymer functionalized polymer of the invention. Boc or the typical amino-protecting groups used in peptide synthesis that can be subsequently removed under mild conditions relative to the product are suitable (see for example Green and Wuts (1991) “Protective Groups in Organic Synthesis,” 2^{nd} ed., Wiley, New York, N.Y.).

In another embodiment, functionalized polymers of the invention are represented by the following formula:

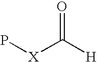

wherein P represents a polymer as previously defined and X represents a spacer group which is optionally present. The spacer group X may be absent or present and if present, functions to connect an aldehyde to the polymer of interest. The spacer group X represents a non-reacting group comprising substituted or unsubstituted, branched or liner, aliphatic or aromatic groups such as phenyl or C₁-C₁₀ alkylene moieties, C₁-C₁₀ alkyl groups or a combination thereof, or an amino acid chain (such as a flexible hinge or loop sequence (see, for example, J. Mol. Biol., 211, 943-958 (1990)), a nucleotide chain, a sugar chain, a lipid chain, or a combination thereof and may contain heteroatoms. In the preferred embodiments, X comprises —CH₂—. Groups present in the additional connecting structure (i.e., spacer groups) adjacent to the aldehyde are not critical; however, a requirement of any spacer group is that it not interfere with the formation of the carbon-nitrogen bond between the aldehyde and its complementary primary amino group. Furthermore, spacer groups should not react in preference to the aldehyde with the primary amino group, nor provide steric hindrance to the reaction, nor deactivate the reactive groups.

The invention also provides methods for inhibiting endothelial cell proliferation in vitro and in vivo using a conjugated kringle peptide fragment as well as methods for treating a human or animal in need of anti-angiogenic therapy comprising administering to such individuals a therapeutically effective amount of a conjugate of a kringle peptide fragment. The invention further provides pharmaceutical compositions comprising a conjugate of a kringle peptide fragment and a pharmaceutically acceptable excipient.

The compounds of the invention, including but not limited to those specified in the examples, possess anti-angiogenic activity. As angiogenesis inhibitors, such compounds are useful in the treatment of both primary and metastatic solid tumors and carcinomas of the breast; colon; rectum; lung; oropharynx; hypopharynx; esophagus; stomach; pancreas; liver; gallbladder; bile ducts; small intestine; urinary tract including kidney, bladder and urothelium; female genital tract including cervix, uterus, ovaries, choriocarcinoma and gestational trophoblastic disease; male genital tract including prostate, seminal vesicles, testes and germ cell tumors; endocrine glands including thyroid, adrenal, and pituitary; skin including hemangiomas, melanomas, sarcomas arising from bone or soft tissues and Kaposi's sarcoma; tumors of the brain, nerves, eyes, and meninges including astrocytomas, gliomas, glioblastomas, retinoblastomas, neuromas, neuroblastomas, Schwannomas and meningiomas; solid tumors arising from hematopoietic malignancies such as leukemias and including chloromas, plasmacytomas, plaques and tumors of mycosis fungoides and cutaneous T-cell lymphoma/leukemia; lymphomas including both Hodgkin's and non-Hodgkin's lymphomas; prophylaxis of autoimmune diseases including rheumatoid, immune and degenerative arthritis; ocular diseases including diabetic retinopathy, retinopathy of prematurity, corneal graft rejection, retrolental fibroplasia, neovascular glaucoma, rubeosis, retinal neovascularization due to macular degeneration and hypoxia; abnormal neovascularization conditions of the eye; skin diseases including psoriasis; blood vessel diseases including hemagiomas and capillary proliferation within atherosclerotic plaques; Osier-Webber Syndrome; myocardial angiogenesis; plaque neovascularization; telangiectasia; hemophiliac joints; angiofibroma; wound granulation; diseases characterized by excessive or abnormal stimulation of endothelial cells including intestinal adhesions, Crohn's disease, atherosclerosis, scleroderma and hypertrophic scars (i.e., keloids) and diseases which have angiogenesis as a pathologic consequence including cat scratch disease (Rochele minalia quintosa) and ulcers (Helicobacter pylori). Another use is as a birth control agent which inhibits ovulation and establishment of the placenta.

The compounds of the present invention may also be useful for the prevention of metastases from the tumors described above either when used alone or in combination with radiotherapy and/or other chemotherapeutic treatments conventionally administered to patients for treating angiogenic diseases and/or in combination with other anti-angiogenic agents. For example, when used in the treatment of solid tumors, compounds of the present invention may be administered with chemotherapeutic agents such as alpha interferon, COMP (cyclophosphamide, vincristine, methotrexate and prednisone), etoposide, mBACOD (methotrexate, bleomycin, doxorubicin, cyclophosphamide, vincristine and dexamethasone), PRO-MACE/MOPP prednisone, methotrexate (w/leucovin rescue), doxorubicin, cyclophosphamide, taxol, etoposide/mechlorethamine, vincristine, prednisone and procarbazine), vincristine, vinblastine, angioinhibins, TNP-470, pentosan polysulfate, platelet factor 4, angiostatin, LM-609, SU-101, CM-101, Techgalan, thalidomide, SP-PG and the like. Other chemotherapeutic agents include alkylating agents such as nitrogen mustards including mechloethamine, melphan, chlorambucil, cyclophosphamide and ifosfamide; nitrosoureas including carmustine, lomustine, semustine and streptozocin; alkyl sulfonates including busulfan; triazines including dacarbazine; ethyenimines including thiotepa and hexamethylmelamine; folic acid analogs including methotrexate; pyrimidine analogues including 5-fluorouracil, cytosine arabinoside; purine analogs including 6-mercaptopurine and 6-thioguanine; and-tumor antibiotics including actinomycin D; the anthracyclines including doxorubicin, bleomycin, mitomycin C and methramycin; hormones and hormone antagonists including tamoxifen and cortiosteroids and miscellaneous agents including cisplatin and brequinar. For example, a tumor may be treated conventionally with surgery, radiation or chemotherapy and conjugated kringle 5 administration with subsequent conjugated kringle 5 administration to extend the dormancy of micrometastases and to stabilize and inhibit the growth of any residual primary tumor. Other anti-angiogenic agents include other kringle peptide fragments of plasminogen such as K1 and K4-5 as well as K2 of prothrombin.

The term “parenteral,” as used herein, refers to modes of administration which include intravenous, intramuscular, intraperitoneal, intrasternal, subcutaneous and intraarticular injection and infusion. Pharmaceutical compositions for parenteral injection comprise pharmaceutically acceptable sterile aqueous or non-aqueous solutions, dispersions, suspensions or emulsions as well as sterile powders for reconstitution into sterile injectable solutions or dispersions just prior to use. Examples of suitable aqueous and non-aqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity may be maintained, for example, by the use of coating materials such as lecithin, by the maintenance of the required particle size in the case of dispersions and by the use of surfactants. These compositions may also contain adjuvants such as preservatives, wetting agents, emulsifying agents and dispersing agents. Prevention of the action of microorganisms may be ensured by the inclusion of various antibacterial and antifungal agents such as paraben, chlorobutanol, phenol sorbic acid and the like. It may also be desirable to include isotonic agents such as sugars, sodium chloride and the like. Prolonged absorption of the injectable pharmaceutical form may be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations may be sterilized, for example, by filtration through a bacterial-retaining filter or by incorporating sterilizing agents in the form of sterile solid compositions which can be dissolved or dispersed in sterile water or other sterile injectable media just prior to use.

Topical administration includes administration to the skin, mucosa and surfaces of the lung. Compositions for topical administration may be prepared as a dry powder which may be pressurized or non-pressurized. In non-pressurized powder compositions, the active ingredient in finely divided form may be used in admixture with a larger-sized pharmaceutically acceptable inert carrier comprising particles having a size, for example, of up to 100 micrometers in diameter. Suitable inert carriers include sugars such as lactose. Desirably, at least 95% by weight of the particles of the active ingredient have an effective particle size in the range of 0.01 to 10 micrometers. Alternatively, a compound of the invention may be injected directly into the vitreous and aqueous humor of the eye.

Compounds of the present invention may also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form may contain, in addition to a compound of the present invention, stabilizers, preservatives, excipients and the like. The preferred lipids are the phospholipids and the phosphatidyl cholines (lecithins), both natural and synthetic. Methods to form liposomes are known in the art.

When used in the above or other treatments, a therapeutically effective amount of one of the compounds of the present invention may be employed in pure form or, where such forms exist, in pharmaceutically acceptable salt form and with or without a pharmaceutically acceptable excipient. A “therapeutically effective amount” of the compound of the invention means a sufficient amount of the compound to treat an angiogenic disease (for example, to limit tumor growth or to slow or block tumor metastasis) at a reasonable benefit/risk ratio applicable to any medical treatment. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of the compound at levels lower than those required to achieve the desired therapeutic effect and to gradually increase the dosage until the desired effect is achieved. Total daily dose of conjugated kringle 5 peptide fragments to be administered locally or systemicaly to a human or other mammal host in single or divided doses may be in amounts, for example, from 0.01 to 5 mg/kg body weight daily and more usually 0.05 to 1 mg/kg body weight. If desired, the effective daily dose may be divided into multiple doses for purposes of administration. Consequently, single dose compositions may contain such amounts or submultiples thereof to make up the daily dose.

It will be understood that agents which can be combined with the compound of the present invention for the inhibition, treatment or prophylaxis of angiogenic diseases are not limited to those listed above, but include, in principle, any agents useful for the treatment or prophylaxis of angiogenic diseases

### Synthetic Processes

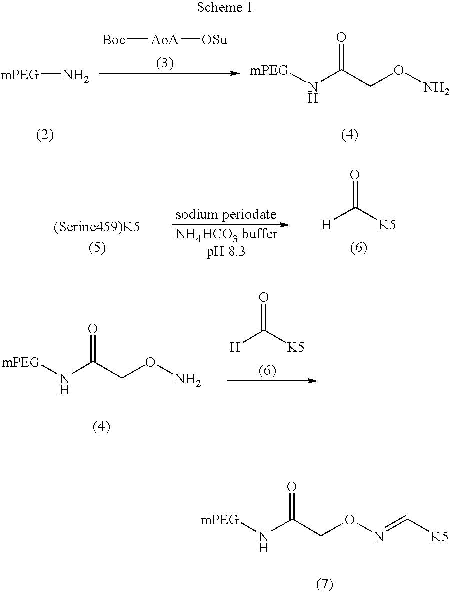

Scheme 1 shows the preparation of one type of polyalkylene glycol conjugate of K5, which can readily be extended to the synthesis of other conjugated kringle peptide fragments. Aminomethoxypolyethylene glycols (2) (5-30 kD) can be condensed with (N-tert-butoxycarbonylamino)oxy)acetyl N-hydroxysuccinimide ester (3) to provide (amino-oxy)acetyl-functionalized polyethylene glycols (4). These can be condensed with aldehyde (6) (formed by the oxidation of (Serine459)K5 (5) with sodium periodate) to form the desired conjugated peptide fragments (7) with varying polyethylene glycol lengths.

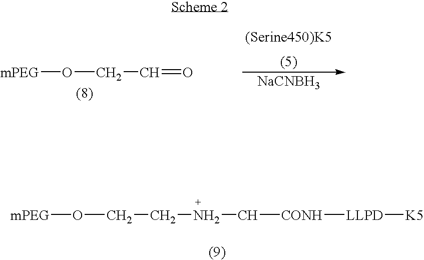

Scheme 2 shows the preparation of an alternate polyethylene glycol conjugate of K5, which can also readily be extended to the synthesis of other conjugated kringle peptide fragments. Methoxypolyethylene glycol aldehyde (8) (5-30 kD) can be condensed with (Serine459)K5 in the presence of a reducing agent such as sodium cyanoborohydride to form the desired conjugated peptide fragments (9) (LLPD-K5=leucine-leucine-proline-aspartic acid-K5) with varying polyethylene glycol lengths.

The following examples will serve to further illustrate the preparation of the novel compounds of the invention:

### EXAMPLE 1

### Construction of pET32-Kan-Ek-SLLPD-K5, pET32-Kan-Ek-SEED-K5, pET32-Kan-Tev-SEED-K5

PCR was employed to generate kringle fusion proteins which comprise a kringle 5 peptide fragment. A 5′ PCR primer coding for an Enterokinase cleavage site Asp-Asp-Asp-Asp-Lys (SEQ ID NO:2) followed by a Ser residue and the K5 N-terminal coding sequences beginning at plasminogen residue 450 and extending to residue 453 of SEQ ID NO:1 was synthesized: TGGGTACCGACGACGACGACAAGTCCCTGCTTCCAGATGTAGAGA (SEQ ID NO:3). A 5′ PCR primer coding for an Enterokinase cleavage site Asp-Asp-Asp-Asp-Lys (SEQ ID NO:2) followed by the K5 N-terminal coding sequences beginning at plasminogen residue 458 and extending through residue 461 of SEQ ID NO:1 was synthesized:

TGGGTACCGACGACGACGACAAGTCCGAAGAAGACTGTATGTTTGGG (SEQ ID NO:4). A 5′ PCR primer coding for a Tobacco Etch Virus TEV protease cleavage site Glu-Asn-Leu-Tyr-Phe-Gln (SEQ ID NO:5) followed by the K5 N-terminal coding sequences beginning at plasminogen residue 458 and extending through residue 461 of SEQ ID NO:1 was synthesized:

TGGGTACCGAAAACCTGTATTTTCAGTCCGAAGAAGACTGTATGTTTGGG (SEQ D3 NO:6). A K5 C-terminal 3′ primer TTATTAGGCCGCACACTGAGGGA (SEQ DD NO:7) also was synthesized. PCR fragments were generated separately using 50 μL of Pfu DNA polymerase buffer (Stratagene®, Lajolla, Calif.), 200 μM each dNTP, 500 μM ATP, 0.5 μM each of one of the appropriate 5′ primers and the 3′ primer and T4 polynucleotide kinase. After incubation at 37° C. for 15 mins. to kinase the primers, Pfu DNA polymerase and approximately 10 ng of DraI-digested vector pHIL-D8 containing K4-K5A plasmid (described previously in U.S. Pat. No. 6,057,122) was added. Using SEQ ID NO:4 and SEQ ID NO:7 as primers, after 1 minute at 94° C., PCR was run with a thermocycle file using 20 cycles of [94° C. 1 sec, 40 sec; 72° C. 1 sec; 50° C. 1 min, 30 sec; 72° C. 1 sec, 4 mins] which resulted in a product that encodes Ek-SEED-K5. Using SEQ ID NOs:3 and 7 and SEQ ID NOs:6 and 7 as primer sets, after 1 minute at 94° C., PCR was run with a thermocycle file using 20 cycles of [94° C. 1 sec, 40 sec; 72° C. 1 sec; 50° C. 2 min, 30 sec; 72° C. 1 sec, 4 mins] which resulted in PCR products that encodes Ek-SLLPD-K5 and Tev-SEED-K5, respectively. The PCR products were then purified over S400-HR spin columns (Amersham Pharmacia Biotech, Piscataway, N.J.).

A pET32-Kan expression vector was constructed in the following manner: pET32a DNA (Novagen; Madison, Wis.) was cut with Bpul 102I+SapI (New England Biolabs; Beverly, Mass.) and the 3426 bp fragment (map positions 81-3507) was purified by agarose gel electrophoresis. pET24d DNA (Novagen, Madison, Wis.) was also cut with Bpul 102I+SapI and the 2341 bp. fragment (map positions 3047-81) containing the Kan^{r} gene was purified by agarose gel electrophoresis. The two fragments were ligated using a rapid ligation kit Roche Molecular Biochemicals; Indianapolis, Ind.) and E. coli BL21 (DE3) competent cells (Novagen; Madison, Wis.) were transformed with the ligation mix and plated onto LB-Kan agar plates (Micro Diagnostics; Lombard, Ill.). Individual colonies were grown up in LB-Kan medium and plasmid DNA purified using midiprep kits (Quiagen; Valencia, Calif.). The DNA sequence of the coding region including the T7 promoter and T7 terminator was verified by DNA sequence analysis.

One μg of pET32-Kan was digested with BglII plus XhoI, then the ends were filled and the DNA phosphatased and purified as described previously (U.S. Pat. No. 6,057,122, supra). One μL (approximately 10 ng) of this pET32-Kan vector and 1 μL of a K5 PCR product were ligated in a volume of 5.25 μL using a Rapid Ligation Kit as described previously. One μL of each ligation mixture was then transformed into 20 μL of HMS174(DE3) competent cells (Novagen, Madison, Wis.). Recombinant cells were selected on LB-Kanamycin agar. Colonies were screened by PCR for the presence of an insert of the correct size and orientation as described previously (U.S. Pat. No. 6,057,122, supra). Correct colonies were then grown up for isolation of plasmid DNA for DNA sequence verification, and for small-scale expression studies as described previously (U.S. Pat. No. 6,057,122, supra).

### EXAMPLE 2

### Construction of pET32-Kan-Tev-SLLPD-K5 and pET32-Kan-Tev-LLPD-K5

Two 5′ PCR primers coding for a TEV protease cleavage site Glu-Asn-Leu-Tyr-Phe-Gln (SEQ ID NO:5), one with and one without a Ser residue, followed by a K5 N-terminal coding sequences beginning at plasminogen residue 450 and extending through residue 453 of SEQ ID NO:1 were synthesized:

TGGGTACCGAAAAACCTGTATTTTCAGTCCCTGCTTCCAGATGTAGAGA (SEQ ID NO:8) and TGGGTACCGAAAACCTGTATTTTCAGCTGCTTCCAGATGTAGAGACTC (SEQ ID NO:9). The C-terminal 3′ primer was prepared as described in Example 1 above. PCR reactions were prepared on ice in thin-walled tubes, then transferred directly to a Perkin-Elmer 480 thermocycler block preheated to 94° C. Using SEQ ID NOs:8 and 7 and SEQ ID NOs:9 and 7 as primer sets, after 1 minute at 94° C., PCRs were run with a thermocycle file using 25 cycles of [94° C. 1 sec, 40 sec; 80° C. 1 sec; 45° C. 5 min, 30 sec; 72° C. 1 sec, 8 mins.], resulting in products encoding Tev-SLLPD-K5 and Tev-LLPD-K5, respectively. Cloning was performed as described in Example 1 above.

### Example 3

### Isolation and Purification of K5 from Fusion Proteins

(a) Isolation of Fusion Protein: Cell paste of Ek-SLLPD-K5, Ek-SEED-K5, Tev-SLLPD-K5, Tev-SEED-K5 or Tev-LLPD-K5 from Examples 1 and 2 was removed from −80° C. storage and mixed with lysis buffer (50 mM Tris, 300 mM NaCl, 1 mM NaN₃, pH 7.9, Hampton Research, Laguna Niguel, Calif.), protease inhibitors (87 μg/mL PMSF, 5 μg/mL aprotinin, 78 μg/mL benzamidine, 1 μg/mL leupeptin, 5 μg/mL phenanthrolin) (SIGMA, St. Louis, Mo.), and Benzonase (20 μL/100 grams cell paste) (EM Industries, Hawthorne, N.Y.). The cells in the suspension were lysed using a Microfluidizer at 11,000 psi (Microfluidics, Newton, Mass.). Typical lysis was >90% as confirmed by microscopy. The supension was clarified by centrifugation (RCF=22000 g for 30 minutes at 4° C.). The supernatant was decanted and mixed with Ni IMAC resin (Probond, Invitrogen Corporation, Carlsbad, Calif.) in a batch method. The resin was washed with lysis buffer to remove non-specifically bound proteins. The fusion protein was eluted in a stepwise gradient of imidazole (50 mM, 100 mM, 250 mM, and 500 mM in lysis buffer. The pure fusion protein was concentrated and the buffer was exchanged by dialysis to cleavage buffer (50 mM Tris, 1 mM NaN₃, pH 7.9) (Hampton Research, Laguna Niguel, Calif.).

(b) Cleavage of Fusion Protein using Enterokinase: The Ek fusion protein solution was made to a final concentration of 1 mM CaCl₂. Enterokinase (EKMax, Invitrogen Corporation, Carlsbad, Calif.) was added to the fusion protein at 0.8163 to 14.3 units per gram of fusion protein as determined by absorbance at 280 nm. The solution was brought to 37° C., and the cleavage reaction was allowed to continue for 20 to 24 hours. The progression of cleavage was monitored by SDS-PAGE under reducing conditions.

(c) Cleavage of Fusion Protein using TEV protease: The Tev fusion protein solution was made to a final concentration of 1 mM DTT (SIGMA, St. Louis, Mo.). Recombinant TEV Protease, (GibcoBRL Life Technologies, Gaithersburg, Md.) was added to the fusion protein at 76.2 to 1330 units per gram of fusion protein as determined by absorbance at 280 nm. The solution was brought to 30° C., and the cleavage reaction was allowed to continue for 20 to 24 hours. The progression of cleavage was monitored by SDS-PAGE under reducing conditions.

(d) Isolation of K5: Cleaved K5 was isolated by mixing the cleavage solution with Ni IMAC resin (Probond, Invitrogen Corporation, Carlsbad, Calif.) in a batch method. The cleavage flow-through was collected and the resin was washed with cleavage buffer. The wash solutions that contained the K5 were combined with the cleavage flow-through, and applied to an anion exchange resin (Q Sepharose fast flow, SIGMA, St. Louis, Mo.). Pure K5 was isolated using a linear NaCl gradient (0 M to 300 mM in cleavage buffer. Cleavage of the fusion proteins with the appropriate enzyme resulted in products referred to hereinafter as SLLPD-K5, SEED-K5, and recombinant LLPD-K5.

### EXAMPLE 4

### Functionalization of the N-Terminus of SLLPD-K5 and Conjugation with Methoxypolythyleneglycol fmPEG, mol. wt. 20,000)

Kringle 5 peptide fragment SLLPD-K5 was functionalized and conjugated with methoxypolyethylene glycol (5-30 kD) essentially as described by Gaertner et al. in Bioconjuxate Chem., 7, 38-44 (1996). The protocol used is described briefly below.

(a) Formation of N-hydroxysuccinimide ester of BOC-AOA: A solution of BOC-amino-oxyacetic acid (BOC-AOA, 1.9 g, purchased from Novabiochem) in ethyl acetate (30 mL) at room temperature was treated with a solution of N-hydroxysuccinimide (1.15 g) in ethyl acetate (30 mL). The resulting mixture was treated with a solution of N,N′-dicyclohexylcarbodiimide (2.06 g) in ethyl acetate (5 mL), stirred for 18 hours, filtered, and concentrated. The concentrate was triturated with diethyl ether (40 mL), filtered, and the resulting solid dried under vacuum to provide 1.2 g of the desired product.

MS m/e306 (M+NH₄)⁺;

¹H NMR δ 1.48 (9H), 2.88 (4H), 4.78 (2H).

(b) Formation of MeO-PEG_{20 kd}-NH—COCH₂—O—NH(AOA-PEG_{20 kd}): A solution of MeO-PEG_{20 kd}-NH₂ (1.5 g, purchased from Rapp Polymere Inc., Tubingen, Germany) in acetonitrile (4.5 mL) at room temperature was treated with Example 3A (50 mg) and 4-methylmorpholine (0.1 mL), stirred for 2 hours, and concentrated. The concentrate was dissolved in water (9 mL),treated with p-alanine (100 mg), and adjusted to pH 9.2 with 2N NaOH. The mixture was dialyzed against water (2 changes of 4 liters) using 3,500 mol. wt. cutoff dialysis tubing, then lyophilized to provide 1.4 g of BOC-AOA-PEG-OMe.

A solution of BOC-AOA-PEG-OMe (1.4 g) in trifluoroacetic acid (TFA, 2.5 mL) at room temperature was allowed to stand for 3 hours, concentrated, treated with water (5 mL), and concentrated again. The concentrate was dissolved in water (7.5 mL) and adjusted to pH 3.0 with 2M NaOH. The solution was then dialyzed (3.5 kD cutoff dialysis tubing) against water at 4° C. and lyophilized. A portion of the resulting solid (29 mg) was dissolved in water (1 mL). The amino-oxy functional group of this solution was estimated spectrophotometrically by condensation with m-nitrobenzaldehyde (MNBA) in 50 mM acetate buffer, pH 4.0. Quantitative reaction from a solution of a weighed amount of MNBA with excess AOA-PEG-OMe showed that the molar increase in absorbance of this aldehyde when converted to the mPEG-oxime is 12,400 at 255 nm. Complete reaction of AOA-PEG-OMe was accomplished in 15 minutes at 60° C. when the aldehyde was 1.0 mM, and in 1.5-2 fold excess over the concentration of AOA-PEG-OMe as estimated by weight of the latter. The 29 mg/mL solution was found to be 0.95 mM (68% active), equivalent to 19 mg/mL aldehyde-reactive polymer, based on a mol wt. of 20,000.

(c) Oxidation of SLLPD-K5: Purified SLLPD-K5 at approximately 1 mg/mL was supplied in Tris-Cl buffer containing 100 mM NaCl, pH 7.8. The protein concentrations of K5, SLLPD-K5, and PEG conjugates of K5 were estimated from optical density at 280 nM, where a 1.0 mg/mL solution (1.0 cm path length, pH range 4-8.5) has OD=1.70.

SLLPD-K5 was dialyzed into 0.1M NH₄CO₃ (pH 7.5-8), then concentrated by filtration through 3 kd cutoff membrane to 3.3 mg/mL. The solution (66.6 mL, 220 mg kringle 5 mol. wt. 10,500) was treated with 1-methionine (50 mg) to provide 5 mM final methionine concentration. The mixture was adjusted to pH 8.6 with concentrated NH₄OH, treated with sodium periodate (18 mg, 4 equivalents), and monitored by reverse phase HPLC (C-8 Waters Symmetry 4.6×250 mm, acetonitrile in water with 0.1% TFA, CH₃CN increasing from 5%-40% over 30 minutes). After 1 hour the mixture was treated with an additional 13.5 mg, stirred for an additional hour, treated with 25 μL of propanediol, stirred for 1 hour, and adjusted to pH 4.8 with 1:1 glacial acetic acid/water. The mixture was dialyzed (3,500 mol wt cutoff membrane) overnight against 50 mM sodium acetate buffer, pH 5.0 (4 L) and analyzed by mass spectrum analysis, which indicated the presence of glyoxylyl kringle 5 (10,516) and its hydrate (10,534).

(d) Condensation of AOA-PEG with GI oxylyl-LLPD-K5: A solution of Example 4(c) (3 mg/mL, 66.7 mL, 19 μmmol) was adjusted to pH 4.0 by drop-wise addition of 1:1 glacial acetic acid/water. The solution was treated with 0.95 mM of Example 4(b) (34 mL, 32 μmmol, 1.68-fold excess over protein) and concentrated to ⅕ of the original volume using a stirred cell with 3,500 mol. wt. cutoff membrane, over a 4 hour period to provide a final protein concentration of approximately 10 mg/mL. The reaction was continued for 24 hours, quenched with 100 mM m-nitrobenzaldehyde in ethanol (0.12 mL), stirred for 30 minutes, and adjusted to pH 7.8 with 2M Tris base to provide the desired product.

mPEG also was conjugated to SEED-K5 essentially as described above. The SLLPD-K5 and SEED-K5 peptides conjugated to methoxypolyethylene glycol are referred to hereinafter as mPEG-SLLPD-K5 and mPEG-SEED-K5.

### EXAMPLE 5

### Generation of PEGylated Kringle Peptide Fragments

Using methods well known in the art (such as, for example, recombinant and synthetic techniques) for generating kringle peptide fragments and/or kringle peptide fragments having a naturally occurring or non-naturally occurring N-terminal serine and/or by following Scheme 1 and the examples above, the following compounds can be prepared:

(a) Kringle 1 (of plasminogen)-methoxypolyethylene glycol conjugate,

(b) Kringles 4-5 (of plasminogen)-methoxypolyethylene glycol conjugate, and

(c) Prothrombin Kringle 2-methoxypolyethylene glycol conjugate.

In addition, conjugates of each of the above-mentioned kringle peptide fragments with other polyalkylene glycols can be synthesized by substituting alternative polyalkylene glycols for methoxypolyethylene glycol in the procedures outlined in Scheme 1 and Examples 1-5.

### EXAMPLE 6

### Endothelial Cell Migration Assay

A primary line of HMVEC (human dermal microvascular endothelial cells) were grown to 80-90% confluency, the cells were washed with phosphate-buffered saline (PBS), then left for 18 hours in minimal media (Eagles balanced salt solution, no supplements) containing 10% fetal calf serum (FCS). The cells were washed with PBS and trypsinized and resuspended at 2-5×10⁶ cells/mL, then loaded with fluorophore by incubation with Calcein AM™ (MOLECULAR PROBES, Eugene, Oreg.) in the dark. Vascular endothelial growth factor (VEGF) in media was placed in wells of 96 well plates, and a filter then placed above these solutions. After centrifugation, re-suspension, and assessment of the extent of labeling, the cells were added to the tops of these wells with varied amounts of rK5 (amino acid position 450 to amino acid 543 of SEQ ID NO:1), purified mPEG-SLLPD-K5 or purified mPEG-SEED-K5. After 4 hours at 37° C., free cells (unmigrated) were wiped from the top filter, and fluorescence measured. Data were derived as a fraction of the number of cells which migrated in control wells containing no inhibitor and are shown in Table 1 as % inhibition. Sem represents standard error of the mean.

TABLE 1
Inhibition of EC Migration by mPEG Kringle 5 Constructs

| Protein | | | mPEG- | | mPEG- | |
|---|---|---|---|---|---|---|
| Concentration | rK5 | | SLLPD-K5 | | SEED-K | |
| (ng/mL) | (% I) | sem | (% I) | sem | (% I) | sem |
| 10 | 73 | 1 | 70 | 15 | 75 | 13 |
| 1 | 47 | 7 | 59 | 10 | 71 | 3 |
| 0.1 | 32 | 12 | 53 | 23 | 65 | 9 |
| 0.01 | 45 | 8 | 68 | 4 | 39 | 15 |
| 0.001 | 19 | 18 | 19 | 12 | 53 | 7 |
| 0.0001 | 8 | 8 | 33 | 21 | 46 | 9 |

As Table 1 shows, mPEG-SLLPD-K5 and mPEG-SEED-K5 inhibited endothelial cell migration to the same, if not to a greater extent, than unpegylated rK5.

### EXAMPLE 7

### Monkey Pharmacokinetics

All samples were diluted in phosphate buffered saline to a concentration of 1.1 mg/mL. Nine cynomolgus monkeys, weighing 3-6 kg, were fasted overnight prior to dosing but were allowed water ad libitum. Food was returned to the monkeys approximately three hours after dosing. During the study the animals were housed individually.

This study was performed in parallel, in three groups of monkeys; each group contained three animals. All animals received a 1 mg/kg (0.91 mL/kg) intravenous dose of compound (either mPEG-SLLPD-K5, mPEG-SEED-K5 or recombinant LLPD-K5) administered as a slow bolus in a saphenous vein. EDTA preserved blood samples (about 3 mL) were withdrawn from a femoral artery or vein of each animal prior to dosing and at 0.1, 0.25, 0.5, 1, 2, 3, 4, 6, 9 and 24 hours after drug administration. The results are shown in Table 2 below and in FIG. 2.

TABLE 2

| | 1 mg/kg IV Dose | | | | |
|---|---|---|---|---|---|
| | T½* | V_{c}⁺ | Vβ\hat{ } | AUC_{0-∞}^{#} | Clₚ** |
| Form | (hr) | (L/kg) | (L/kg) | (μg · hr/mL) | (L/hr · kg) |
| r-LLPD-K5 | 0.19 | 0.033 | 0.033 | 9.85 | 0.11 |
| mPEG-SEED-K5 | 5.05 | 0.044 | 0.057 | 150.20 | 0.0078 |
| mPEG-SLLPD-K5 | 5.53 | 0.031 | 0.067 | 126.66 | 0.0081 |

*t½ = half-life in circulation
⁺Vc = volume parameter
\hat{ }V|3 = volume parameter
^{#}AUC_{0-∞} = area under the curve
**Clₚ = clearance rate

As Table 2 shows, both mPEG-SEED-K5 and mPEG-SLLPD-K5 displayed a 27×longer half-life (t½) than recombinant LLPD-K5 and a 13×slower clearance rate (Clp) than non-pegylated K5 when dosed intravenously in monkeys.

### EXAMPLE 8

### Conjugation of the N-Terminus of SLLPD-K5 with Methoxypolyethyleneglycol Aldehyde (PEG-Aldehyde, mol. wt. 20,000)

Kringle 5 peptide fragment SLLPD-K5 was functionalized and conjugated with methoxypolyethylene glycol (5-30 kD) essentially as described in J. Pharmaceut. Sci., 87, 1446-1449 (1998). The protocol used is described briefly below.

(a) Titration of Methoxypolyethyleneglycol aldehyde: A mixture of 2 mM methoxypolyethyleneglycol aldehyde (200 μL of 40 mg/mL in H₂O) was treated with 10 μM p-nitrophenylhydrazine (200 μL, 15.3 mg in 10 mL ethanol) and incubated at room temperature for 24 hours. A 50 |μL aliquot was diluted to 1.0 mL with 100 mM HCl and measured for absorbance at 405 nm at t=0 and t=24 hrs. The change in absorbance (λ₄₀₅=0.64, t=24 hrs) was compared to that of a 50 μM standard of the p-nitrophenylhydrazone of 3-phenylpropanal (λ₄₀₅=0.67) indicating 96% aldehyde content compared to that predicted by weight.

(b) Condensation of Methoxypolyethyleneglycol aldehyde and SLLPD-K5: A mixture of SLLPD-K5 (300 mg) in 50 mM sodium acetate (21.7 mL, pH 4.7) was treated with methoxypolyethyleneglycol aldehyde (1.14 g, 0.0572 mmol, purchased from Shearwater) and 5M sodium cyanoborohydride in 1M NaOH (43.4 μL, 0.217 mmol). The mixture was incubated at room temperature for 48 hours while the reaction's progress was monitored by HPLC (Waters C8 Symmetry column, 4.6×150 mm; solvent gradient: 5% to 40% acetonitrile/H20 containing 0.1% TFA in 30 minutes). The mixture was treated with additional methoxypolyethyleneglycol aldehyde (200 mg) and sodium cyanoborohydride (43.4 μL), and incubated for an additional 24 hours at room temperature.

(c) Purification of mPEGylated-K5 Formed by Reductive Amination: mPEGylated-K5 was stored at 3-7 mg/mL in 50 mM Tris buffer, 100 mM NaCl, and 1 mM NaN₃ at pH 7.7. Pegylated K5 reaction mixtures were concentrated to approximately 10 mg/mL and dialyzed into a buffer containing 25 mM sodium acetate at pH 5.0 (buffer A) for chromatography. Chromatography was performed on a Tosoh Biosep (Montgomeryville, Pa.) TSK-SP5PW column using 25 mM sodium acetate buffer at pH 5.0. A gradient to buffer A containing 200 mM NaCl at pH 5.0 (buffer B) was generated using a Pharmacia FPLC system to elute the protein. Different sized columns were used to handle different sample loads, but the most commonly used was TSK-SP5PW-HR (2×15 cm). Flow rates were adjusted to match column sizes and varied from 1.0 to 2.5 mL/min. Columns were equilibrated in buffer A and the sample, previously dialyzed into buffer A, was loaded. The column was then washed with up to three column volumes of the buffer A to remove excess mPEG-aldehyde, its removal followed by absorbance at 280 mM. After column washing, a gradient was initiated over 2 to 3 column volumes from buffer A to 25% buffer B (50 mM NaCl). The gradient was put on hold at 50 mM NaCl until the absorbance was restored to baseline. At that point the gradient was increased to 100% buffer B (200 mM NaCl) to elute un-reacted K5. Three peaks eluted in the 50 mM NaCl “hold gradient” stage. SDS Polyacrylamide gel electrophoresis (SDS-PAGE) analysis indicated the first (minor) peak to be about 90,000 MW (presumably di-mPEGylated product). A second minor peak eluted at about 50,000 Da and separated from the third (major) peak also traveling around 50,000 Da on the gel. Fractions from the major peak were pooled by SDS PAGE. The major peak (80-90% of the total) was further characterized for purity and identity by the methods below and was judged to be the N-terminal mono-mPEGylated K5.

Methods used to characterize N-terminal mono-mPEGylated K5:

1. Mass Spectrometry;

2. Analytical Chromatography on Mono-S (HR);

3. Analytical Chromatography on Mono-Q (HR);

4. Hydrophobic Interaction on TSK Phenyl 5PW;

5. Gel Filtration on Superdex 75 (HR);

6. Cyanogen Bromide Cleavage;

7. N-Terminal Sequencing; and

8. Amino Acid Composition Analysis.

### EXAMPLE 9

### Generation of mPEGylated Kringle Peptide Fragments

Using methods well known in the art (such as, for example, recombinant and synthetic techniques) for generating kringle peptide fragments and/or kringle peptide fragments having a naturally occurring or non-naturally occurring N-terminal serine and/or by following Scheme 2 and the example above, the following compounds can be prepared:
(a) Kringle 1 (of plasminogen)-methoxypolyethylene glycol conjugate
(e) Kringles 4-5 (of plasminogen)-methoxypolyethylene glycol conjugate, and
(f) Prothrombin Kringle 2-methoxypolyethylene glycol conjugate.

In addition, conjugates of each of the above-mentioned kringle peptide fragments with other polyalkylene glycols can be synthesized by substituting alternative polyalkylene glycols for methoxypolyethylene glycol in the procedures outlined in Scheme 2 and Example 8.

### EXAMPLE 10

### Monkey Pharmacokinetics

Intravenous dosing was done in cynomolgus monkeys at 1 mg/kg (n=3). Plasma concentrations were determined by Western blot analysis. The results are shown in Table 3.

TABLE 3
Plasma Conc. (μg/ml)

| | | 20 Kd | 20 Kd |
|---|---|---|---|
| Time | rK5 | Oxime PEG K5 | Reductive |
| (hours) | (sem) | (sem) | Amination mPEG K5 (sem) |
| 0.1 | 20.6(1.44) | 30.2(1.13) | 30.1(2.62) |
| 0.25 | 14.34(1.97) | 27.4(1.12) | 32.0(1.84) |
| 0.5 | 6.38 | 26.3(1.2) | 33.64(1.24) |
| 1.0 | 3.47 | 22.12(2.66) | 31.0(0.68) |
| 2 | 0.79 | 17.23(0.82) | 29.64(0.93) |
| 3.0 | — | 10.89(0.25) | 23.93(0.37) |
| 4.0 | — | 9.26(2.1) | 20.84(0.47) |
| 6.0 | — | 5.98(1.3) | 16.1(1.85) |
| 9.0 | ND | 2.29(0.1) | 11.57(1.96) |
| 24.0 | ND | 0.7(0.26) | 1.69(0.46) |
| 36.0 | ND | ND | 0.73(0.15) |

sem = standard error of the mean
ND = not done

As Table 3 shows, mPEGylation at the N-terminus with 20 kD mPEG dramatically decreases the clearance from plasma in monkeys as compared to non-mPEGylated K5. mPEGylation accomplished via reductive amination results in slower clearance of the peptide as compared to mPEGylation by oxime formation.

What is claimed is:

1. A conjugated kringle peptide fragment consisting of a functionalized kringle peptide fragment chemically coupled to a functionalized polymer.

2. The conjugated kringle peptide fragment of claim 1 wherein the N-terminus of the functionalized kringle peptide fragment is conjugated to the functionalized polymer through an oxime bond or through a carbon-nitrogen single bond.

3. The conjugated kringle peptide fragment of claim 2 wherein the functionalized kringle peptide fragment consists essentially of a kringle peptide fragment selected from the group consisting of kringle 1 of plasminogen, kringle 5 of plasminogen, kringles 4-5 of plasminogen, and kringle 2 of prothrombin.

4. The conjugated kringle peptide fragment of claim 3 wherein the kringle peptide fragment is kringle 5 of plasminogen.

5. The conjugated kringle peptide fragment of claim 4 wherein the kringle 5 peptide fragment has substantial sequence homology to a plasminogen fragment selected from the group consisting of human, murine, bovine, canine, feline, Rhesus monkey, and porcine plasminogen.

6. The conjugated kringle peptide fragment of claim 4 wherein the functionalized polymer consists essentially of a polymer which is a polyalkylene glycol.

7. The polymer of claim 6 wherein the polyalkylene glycol is selected from the group consisting of straight, branched, disubstituted, or unsubstituted polyalkylene glycol, polyethylene glycol homopolymers, polypropylene glycol homopolymers, and copolymers of ethylene glycol with propylene glycol, wherein said homopolymers and copolymers are unsubstituted or substituted at one end with an alkyl group.

8. The polymer of claim 7 wherein the polyalkylene glycol is polyethylene glycol (PEG) or methoxypolyethylene glycol (mPEG).

9. The polymer of claim 8 wherein the polyalkylene glycol is methoxypolyethylene glycol (mPEG) and said polyethylene glycol has a molecular weight of about 5,000 to about 40,000.

10. The polyethylene glycol of claim 9 wherein said molecular weight is from about 10,000 to about 20,000.

11. The conjugated kringle peptide fragment of claim 3 wherein the kringle peptide fragment is kringles 4-5 of plasminogen.

12. The conjugated kringle peptide fragment of claim 11 wherein the kringles 4-5 fragment has substantial sequence homology to a plasminogen fragment selected from the group consisting of human, murine, bovine, canine, feline, Rhesus monkey, and porcine plasminogen.

13. The conjugated kringle peptide fragment of claim 11 wherein the functionalized polymer consists essentially of a polymer which is a polyalkylene glycol.

14. The polymer of claim 13 wherein the polyalkylene glycol is selected from the group consisting of straight, branched, disubstituted, or unsubstituted polyalkylene glycol, polyethylene glycol homopolymers, polypropylene glycol homopolymers, and copolymers of ethylene glycol with propylene glycol, wherein said homopolymers and copolymers are unsubstituted or substituted at one end with an alkyl group.

15. The polymer of claim 14 wherein the polyalkylene glycol is polyethylene glycol (PEG) or methoxypolyethylene glycol (mPEG).

16. A pharmaceutical composition comprising a conjugated kringle peptide fragment of claim 4 in combination with a therapeutically acceptable carrier.

17. A pharmaceutical composition comprising a conjugated kringle peptide of claim 11 in combination with a therapeutically acceptable carrier.

18. A method of treating a disease in a patient in need of anti-angiogenic therapy comprising administering to a human or animal a therapeutically effective amount of a conjugated kringle peptide of claim 4.

19. The method of claim 18 wherein the disease is selected from the group consisting of cancer, arthritis, macular degeneration, and diabetic retinopathy.

20. The method of claim 19 wherein the disease is cancer.

21. The method of claim 20 wherein the disease is selected from primary and metastatic solid tumors, carcinomas, sarcomas, lymphomas, psoriasis, and hemagiomas.

22. A method of treating a disease in a patient in need of anti-angiogenic therapy comprising administering to a human or animal a therapeutically effective amount of a conjugated kringle peptide of claim 11.

23. The method of claim 22 wherein the disease is selected from the group consisting of cancer, arthritis, macular degeneration, and diabetic retinopathy.

24. The method of claim 23 wherein the disease is cancer.

25. The method of claim 24 wherein the disease is selected from primary and metastatic solid tumors, carcinomas, sarcomas, lymphomas, psoriasis, and hemagiomas.

26. A method of inhibiting endothelial cell proliferation in an individual comprising administering to said individual an effective amount of a conjugated kringle peptide fragment of claim 4.

27. A method of inhibiting endothelial cell proliferation in an individual comprising administering to said individual an effective amount of a conjugated kringle peptide fragment of claim 11.

28. A method of inhibiting endothelial cell proliferation in vitro comprising administering to an endothelial cell an effective amount of a conjugated kringle peptide fragment of claim 4.

29. A method of inhibiting endothelial cell proliferation in vitro comprising administering to an endothelial cell an effective amount of a conjugated kringle peptide fragment of claim 11.


SEQUENCE LISTING (7 sequences)

SEQ ID NO: 1 - protein, 791 aa, Homo sapiens
EPLDDYVNTQGASLFSVTKKQLGAGSIEECAAKCEEDEEFTCRAFQYHSKEQQCVIMAENRKSSIIIRMRDVVLFEKKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 2 - protein, 5 aa, Artificial Sequence
  Enterokinase Cleavage Site
DDDDK

SEQ ID NO: 3 - DNA, 45 bp, Artificial Sequence
  Forward Primer
tgggtaccgacgacgacgacaagtccctgcttccagatgtagaga

SEQ ID NO: 4 - DNA, 47 bp, Artificial Sequence
  Forward Primer
tgggtaccgacgacgacgacaagtccgaagaagactgtatgtttggg

SEQ ID NO: 5 - protein, 6 aa, Artificial Sequence
  TEV Cleavage Site
ENLYFQ

SEQ ID NO: 6 - DNA, 23 bp, Artificial Sequence
  Reverse Primer
ttattaggccgcacactgaggga

SEQ ID NO: 7 - DNA, 48 bp, Artificial Sequence
  Forward Primer
tgggtaccgaaaacctgtattttcagctgcttccagatgtagagactc